(19) United States
(12) Patent Application Publication
(10) Pub. No.: US 2017/0226480 A1
(43) Pub. Date: Aug. 10, 2017

(54) TREATMENT OF INFLAMMATORY AND DYSIMMUNE RESPONSE

(71) Applicants: UNIVERSITE DE BOURGOGNE, Dijon Cedex (FR); CHU DE DIJON, Dijon Cedex (FR)

(72) Inventors: Bernard BONNOTTE, Dijon (FR); Nona JANIKASHVILI, Tbilisi (GE)

(73) Assignees: UNIVERSITE DE BOURGOGNE, Dijon Cedex (FR); CHU DE DIJON, Dijon Cedex (FR)

(21) Appl. No.: 15/127,053

(22) PCT Filed: Mar. 13, 2015
(86) PCT No.: PCT/EP2015/055331

§ 371 Date: Sep. 19, 2016

(30) Foreign Application Priority Data
Mar. 19, 2014 (FR) 14 52286

(51) Int. Cl. C12N 5/0789; A61K 35/15

(52) U.S. Cl. CPC C12N 5/0647; A61K 35/15; C12N 2501/2306; C12N 2501/22; A61K 2035/124

(57) ABSTRACT

A drug and, more particularly, to a drug for treating inflammatory and dysimmune response. The present invention also relates to a drug for treating graft-versus-host disease. Thus, the present invention relates in particular to a cell expressing CD33, CD11b, CD14, CD163, CD206, HLA-DR, CD44, CD31, CCR5 and CD105.

### FIELD OF THE INVENTION

The present invention relates to a medicinal product and more particularly a medicinal product for inhibiting or reducing immune and inflammatory response. The present invention also relates to a medicinal product for treating diseases for which the immune or inflammatory response is harmful for the patient such as graft-versus-host disease, autoimmune diseases and autoinflammatory diseases.

### PRIOR ART

Immune system and inflammation regulation is one of the therapeutic aims to be achieved to prevent graft-versus-host disease (GvHD) and treat autoimmune and autoinflammatory diseases. GvHD is a complication of transplants caused by the transplantation of allogenic haematopoietic cells. At the present time, the main strategy for combating the onset of GvHD or for treating autoimmune and autoinflammatory diseases consists of using immunosuppressants (corticosteroids, chemotherapy) to induce systemic immunosuppression in the patient. However, these immunosuppressant treatments expose the patient to possible infections and to recurrences of the patient's blood disease. Novel strategies are currently under study, notably the use of suppressor cells such as regulatory T cells (Treg) but, to date, the efficacy of this therapy has not been demonstrated in phase II/III clinical trials. A further cell type known under the acronym MDSC (for “myeloid derived suppressor cells”) is also involved in the negative regulation of the immune system. This cellular subtype is rare or absent in healthy subjects and detected in pathological cases and notably in cases of cancer. Injecting these cells has made it possible in animals to promote post-transplantation graft tolerance but the rarity of these cells makes the clinical use thereof difficult. The present invention proposes the use of a novel cellular subtype of myeloid suppressor cells generated from circulating cells isolated from patients' blood and suitable for use for treating autoimmune and autoinflammatory diseases and graft-versus-host disease. The present invention also relates to a method for preparing, ex vivo, this immunosuppressor cell population.

### SUMMARY OF THE INVENTION

As such, the present invention notably relates to a cell expressing CD33, CD11b, CD14, CD163, CD206, HLA-DR, CD44, CD31, CD105 and CCR5.

The applicants were able to generate, ex vivo, these cells, characterise same by studying the expression of the molecules cited above. The applicants were also able to demonstrate the benefit of these cells within the scope of the treatment of certain diseases wherein the neutralisation of the immune system of the patient to be treated is indicated.

Within the scope of the present invention, the term “cell” refers to a natural or recombinant eukaryotic cell. Preferentially, said cell is a human cell and even more preferentially said cell derives from a haematopoietic stem cell. The term “derive” is intended to signify that said cell is obtained, directly or indirectly, from the division of a haematopoietic stem cell. The various markers cited within the scope of the present invention are well known to those skilled in that art. Preferably, said markers denote any one of the human isoforms of said markers. The CD (for cluster of differentiation) nomenclature, notably used within the scope of this application, was proposed and established by the first international workshop and conference on human leukocyte antigens, held in Paris in 1982. The nomenclature in question is maintained by the HCDM and can notably be viewed on the association's website (www.hcdm.org).

Within the scope of the present invention, the term “expresses” is intended to indicate that the cell according to the invention produces the cited proteins. More particularly, when said proteins are membrane proteins, the term “expresses” signifies that said protein is expressed at the cell membrane of said cell. When said protein is a soluble protein, the term “expresses” is intended to signify that said protein is expressed towards the extracellular domain.

According to one particularly preferred embodiment, the cell according to the invention expresses CD33, CD11b, CD14, CD163, CD206, HLA-DR, CD44, CD31, CD105 and CCR5. According to one preferred embodiment, the cell according to the invention does not express the following molecules: CD1a, CD80, CD86, CD16, CD56, CD3, CD19, CD66b, CCR7 and PDL1.

According to one preferred embodiment, the cell according to the invention expresses CCL2, and IL-6. According to one preferred embodiment, the cell according to the invention does not express the following: IL-4, IL-5, IL-12p70, TNF-α, IL-1β, CCL20, IFNγ, granzyme B, FasL soluble, TGF-β.

The present invention also relates to a cell according to the invention for use as a medicinal product. The possible use as a medicinal product of the cell according to the invention was notably demonstrated in an experimental model of graft-versus-host disease and may be extended to all disease wherein temporary neutralisation of the immune system may be sought. As such, the present invention also relates to the use of the cell according to the invention for treating graft-versus-host disease, autoinflammatory diseases such as giant cell arteritis (Horton disease), rheumatoid arthritis, autoimmune diseases and transplant rejection. The present invention also relates to a composition comprising a cell according to the invention and a pharmaceutically acceptable vehicle. Of the pharmaceutically acceptable carriers, mention may be made of physiological saline solution, PBS, glucose 5%, RPMI.

The present invention also relates to a cell according to the invention for inducing an increase in CD8 regulatory T cells.

Within the scope of the present invention, the term “increase” refers to an increase in proliferation.

The present invention also relates to a cell according to the invention for inducing inhibition of the proliferation of effector T lymphocytes.

The composition according to the invention may further comprise any drugs required within the scope of the envisaged treatment. Preferentially, said composition comprises between 1×10⁷ and 1×10⁸ cells per injection and a number of injections of 1 to 20 injections according to the tolerance and response. The cells according to the invention may be injected into the patient by any routes known to those skilled in the art. Of these, the intravenous route is preferred.

The present invention also relates to a method for preparing a cell according to the invention comprising the steps consisting of:
(i) culturing monocytes in the presence of IL-6 and GM-CSF.
(ii) isolating from the cells obtained following the preceding step, the cells expressing CD33.

According to one alternative embodiment of the present invention, step (ii) may be omitted.

According to one preferred embodiment of the invention, step (i) is performed in the absence of any other chemokines, cytokines and human growth factors.

According to one preferred embodiment of the invention, the monocytes are cultured in step (i) in the absence of IL-4.

According to a further more preferred embodiment of the invention, the monocytes are cultured in step (i) in the absence of IL-1, IL-3, TNF, SCF, EPO and IFN-g.

According to a further more preferred embodiment of the invention, the monocytes are cultured in step (i) in the absence of IFNγ, IL-2 and a chemokine or a combination of chemokines chosen from among CCL2, CCL3, CCL4, CCL8 and CXCL10.

According to one preferred embodiment of the invention, the method according to the invention does not comprise additional steps.

The monocytes are preferentially obtained from the peripheral blood of the patient to be treated, or the blood of allogenic healthy volunteers.

The monocytes may be obtained preferentially using two techniques, either after magnetic isolation of the cells expressing CD14, or from CD34⁺ cells isolated from peripheral blood by magnetic sorting and multiplied during culture in the presence of “CD34 expansion medium” and then differentiated into monocytes during culture in the presence of M-CSF.
(ii) after 7 days of culture, isolating by magnetic sorting from cells obtained following the preceding step, the cells expressing CD33.

Those skilled in the art know the techniques for isolating cells according to the phenotypes thereof well. Said techniques usually use specific, optionally labelled, antibodies for said phenotypes followed by a technique for separating the cells having bound with said antibodies from other cells.

According to one preferred embodiment of the invention, the preparation method according to the invention is characterised in that step (ii) consisting of isolating the cells expressing CD33 is performed via a cell sorter.

According to one preferred embodiment of the invention, the preparation method according to the invention is characterised in that step (ii) consisting of isolating the cells expressing CD33 is performed via magnetic beads.

Step (i) consisting of culturing monocytes in the presence of IL-6 and GM-CSF may be implemented under the culture conditions usually used for this type of cell. Preferentially, the cells are maintained at 37° C. and 5% CO₂ in a suitable culture medium. Preferentially, said culture medium is RPMI 1640. According to one preferred embodiment of the invention, the preparation method according to the invention is characterised in that the IL-6 present in the culture medium in step (i) is between 5 and 15 ng/ml and particularly preferentially between 8 and 12 mg/ml. According to one preferred embodiment of the invention, the preparation method according to the invention is characterised in that the GM-CSF present in the culture medium in step (i) is between 5 and 15 ng/ml and particularly preferentially between 8 and 12 ng/ml. The concentration of GM-CSF and IL-6 indicated is the initial concentration in the culture medium at the time of the contact thereof with the cells. Advantageously, said culture medium is replaced regularly every 2 or 3 days. According to one preferred embodiment of the invention, the preparation method according to the invention is characterised in that step (i) is performed for a period between 4 and 10 days and particularly preferentially of 7 days. According to one preferred embodiment of the invention, the preparation method according to the invention is characterised in that step (i) is performed at 37° C.

### DESCRIPTION OF EMBODIMENTS

### Materials and Methods

Preparation of the Cells According to the Invention

Peripheral blood mononuclear cells were isolated in healthy donors or in patients to be treated using two techniques. The first technique consists of centrifuging peripheral blood on Ficoll gradient, retrieving the PBMC (peripheral blood mononuclear cells), and isolating the monocytes by magnetic sorting using anti-CD14 antibody coupled with a magnetic bead. The second consists of isolating, from peripheral blood, cells expressing CD34 by magnetic sorting, and culturing these cells in the presence of medium promoting the multiplication thereof (CD34 expansion medium), and differentiating same by culturing same in the presence of M-CSF.

The monocytes were then cultured at a concentration of 5.10⁶ cells/ml in RPMI 1640, supplemented with 10% Foetal calf serum, 10 ng/ml GM-CSF and 10 ng/ml IL-6 for 7 days, the medium being replaced every 3 days. The cells according to the invention were then purified, after labelling with a specific CD33 marker, via a cell sorter.
Cell Proliferation Test

T lymphocytes, CD4+CD25− T lymphocytes and CD4+CD25+T lymphocytes were labelled with the “Cell trace Violet cell proliferation kit” (Cell Trace, Carlsbad, Calif.). The labelled cells are cultured in the presence of beads coated with anti-CD3/anti-CD28 (Dynabeads, Invitrogen, Cergy Pontoise, France) with or without the cells according to the invention. The proliferation of the T lymphocytes was detected by flow cytometry.
Morphological Analysis

The morphological analysis of the cells according to the invention was performed after Wright/Giemsa labelling.
Cytokine Assay The concentration of IFN-γ, TNF-α, IL-10, IL-6 and TGF-β was determined in the culture supernatant of the cells cultured using the ELISA technique.
Animal Model of Graft-Versus-Host Disease

NOD/SCID/IL2Rγc-/- mice (Jackson Laboratory), aged 8 to 12 weeks, received peripheral blood mononuclear cells intravenously (20.10⁶ cells per mouse) with or without cells according to the invention (5.10⁶ cells per mouse). The cells are mixed just before injection. The signs of graft-versus-host disease were detected blind every 3 days.
Histological Analysis of Lesions of Graft-Versus-Host Disease

The organs removed from the treated mice were fixed in formaldehyde and included in paraffin. Sections of 5 μm are prepared and stained with eosin and haematoxylin.

### Results

The cells obtained in this way referred to as HuMoSC have the physical, phenotypic and functional characteristics as described below.
Physical Nature of the Cells According to the Invention

After staining with Wright/Giemsa stain, the HuMoSC cells appear as a homogeneous population of large mononuclear cells with a basophilic cytoplasm.
Phenotype of the Cells According to the Invention

The cells according to the invention have a CD33⁺CD11b⁺CD14⁺CD163⁺CD206⁺ HLA-DR⁺CD44⁺CD31⁺CD105⁺ CCR5⁺ phenotype, and weakly expressing CCR6.

The cells according to the invention do not express CD1α, CD80, CD86, CD16, CD56, CD3, CD19 and CCR7.
Effect of the Cells According to the Invention on T Lymphocyte Proliferation

Stimulated autologous T lymphocytes were co-cultured with the cells according to the invention with a ratio of 2 T lymphocytes/cells according to the invention. It was observed that the T lymphocytes, stimulated and co-cultured with the cells according to the invention, proliferate less than T lymphocytes cultured alone. A separate analysis of the T lymphocytes demonstrated that the proliferation of CD4+ T lymphocytes and CD8+ T lymphocytes was also inhibited by the cells according to the invention. Moreover, the antiproliferative effect of the cells according to the invention is also observed on the autologous T lymphocytes and on the allogenic T lymphocytes. The T lymphocytes co-cultured with the cells according to the invention do not express the CD25+ marker unlike the T lymphocytes cultured alone. The analysis of the culture supernatants of the various samples made it possible to demonstrate that the cells according to the invention inhibit the production of proinflammatory cytokine (INF-γ and TNF-α) by T lymphocytes. As such, the cells according to the invention are characterised in that they inhibit cellular activation and cellular proliferation of autologous and allogenic CD4+ and CD8+ T lymphocytes and the cytokine secretion thereof.
The Suppressant Effect of the Cells According to the Invention is STATS-Dependent

The suppressant effect of the cells according to the invention is not dependent on direct cell-to-cell contacts but on one or more soluble factors. Pre-treating the cells according to the invention with an inhibitor of the phosphorylated form of STATS induces loss of the suppressant effect, reduction of CCL2 and IL-6 secretion without interfering with the viability of the cells according to the invention.
The Cells According to the Invention Protect Against the Onset of Graft-Versus-Host Disease

The mice having received human peripheral blood cells develop the clinical signs of graft-versus-host disease between 20 and 30 days post-injection and die before the 50^{th} day post-injection. On the other hand, the mice having received a co-injection of human peripheral blood cells with the cells according to the invention merely exhibit slight signs of graft-versus-host disease and survive this injection. In particular, histological lesions of GvHD in the liver are markedly reduced in the group receiving the cells according to the invention
Effect of the Cells According to the Invention on Regulatory T Lymphocytes.

Regulatory T lymphocytes represent a population of suppressor cells capable of promoting specific alloantigen tolerance.

The mice having received the cells according to the invention exhibit a greater quantity of CD8+ T lymphocytes expressing FoxP3 compared to the control mice.

The same results were observed in vitro after co-culture of a total T lymphocyte population with the cells according to the invention.

1-17. (canceled)

18. Cell comprising it expresses CD33, CD11b, CD14, CD163, CD206, HLA-DR, CD44, CD31, CD105 and CCR5.

19. Cell according to claim 18, wherein it does not express one or a plurality of molecules chosen in the group comprising CD1a, CD80, CD86, CD16, CD56, CD3, CD19, CD66b, CCR7 and PDL1.

20. Cell according to claim 18, wherein it expresses one or a plurality of molecules chosen in the group comprising CCL2 and IL-6.

21. Cell according to claim 18, for use as a medicinal product.

22. Cell according to claim 18, for treating graft-versus-host disease, autoinflammatory diseases, giant cell arteritis (Horton disease), rheumatoid arthritis, autoimmune diseases and transplant rejection.

23. Cell according to a claim 18, for inducing an increase in CD8 regulatory T cells.

24. Cell according to claim 18, for inducing inhibition of the proliferation of effector T lymphocytes.

25. Composition comprising a cell according to claim 18 and a pharmaceutically acceptable vehicle.

26. Method for preparing a cell according to claim 18, comprising the step consisting of:
(i) culturing monocytes in the presence of IL-6 and GM-CSF.

27. Method for preparing a cell according to claim 26, comprising the steps consisting of:
(i) culturing monocytes in the presence of IL-6 and GM-CSF.
(ii) isolating from the cells obtained following the preceding step, the cells expressing CD33.

28. Preparation method according to claim 26, wherein the cells deriving from at least one haematopoietic cell are PBMC.

29. Preparation method according to claim 27, wherein the step consisting of isolating in the cells expressing CD33 is performed via a cell sorter.

30. Preparation method according to claim 27, wherein the step consisting of isolating the cells expressing CD33 is performed via magnetic beads.

31. Preparation method according to claims 26, wherein the IL-6 present in the culture medium in step (i) is between 5 and 15 ng/ml.

32. Preparation method according to claim 26, wherein the GM-CSF present in the culture medium in step (i) is between 5 and 15 ng/ml.

33. Preparation method according to claim 26, wherein step (i) is performed for a period between 4 and 10 days.

34. Preparation method according to claim 26, wherein step (i) is performed at 37° C.
